(19) United States
(12) Patent Application Publication
(10) Pub. No.: US 2022/0133130 A1
(43) Pub. Date: May 5, 2022

(54) ENDOSCOPE HAVING DISTAL PIVOT MECHANISM AND FINE ADJUSTMENT

(71) Applicant: Dr. Konstantin Bob, Weinheim (DE)

(72) Inventor: Konstantin Bob, Weinheim (DE)

(21) Appl. No.: 17/427,326

(22) PCT Filed: Jan. 30, 2020
(86) PCT No.: PCT/EP2020/052351

§ 371 Date: Jul. 30, 2021

(30) Foreign Application Priority Data
Feb. 1, 2019 (DE) 10 2019 102 599.0

(51) Int. Cl. A61B 1/005; A61B 1/00; A61B 1/018; A61B 1/31

(52) U.S. Cl. CPC A61B 1/0055; A61B 1/00098; A61B 1/31; A61B 1/018; A61B 1/0057; A61B 1/00174

(57) ABSTRACT

An endoscope having an endoscope head arranged on the distal end of the endoscope, preferably on the distal end portion of an actively curvable shaft portion. The endoscope has at least one optics device for imaging, and a working channel that extends in or on the endoscope along a longitudinal direction. The working channel has a working channel exit arranged on or in the endoscope head with a defined alignment relative to the optics device. The endoscope also has a pivot mechanism arranged between the distal shaft portion and the endoscope head. The pivot mechanism holds the endoscope head together with the working channel such that the endoscope head is foldable or bendable.

### CROSS-REFERENCE TO RELATED APPLICATIONS

This application is the United States national phase entry of International Application No. PCT/EP2020/052351, filed Jan. 30, 2020, and claims the benefit of priority of German Application No. 10 2019 102 599.0, filed Feb. 1, 2019. The contents of International Application No. PCT/EP2020/052351 and German Application No. 10 2019 102 599.0 are incorporated by reference herein in their entireties.

### FIELD

The present invention relates to an endoscope, in particular a combined colonoscope/duodenoscope comprising an endoscope head which is preferably arranged on an actively curvable, distal end portion of an endoscope shaft and which has at least one optics device for imaging and a working channel which extends in or on the endoscope along the longitudinal direction thereof and the distal working channel exit of which is arranged on or in the endoscope head, the endoscope head having a pivot mechanism or a pivot mechanism is associated with the endoscope head, the pivot radius of which differs from (or is smaller than) the radius of curvature of the actively curvable, distal end portion (hereinafter referred to as deflecting), if present.

### BACKGROUND

Endoscopes are medical working apparatus for the visual exploration of cavities in a patient's body. They basically have optical equipment at the distal end of the endoscope (also called endoscope head) facing away from the user and optionally a working channel that extends from a proximal endoscope section (facing the user) or extracorporeal endoscope handle through a flexible/flexurally stiff or rigid endoscope shaft (adjoining thereto) to the endoscope head and allows the extracorporeal insertion and use of a medical instrument such as forceps, scissors, needles, snares, knifes and the like.

Such endoscopes can optionally be provided with additional functions, for example by placing a cap or sleeve radially outside on the endoscope head on the distal endoscope end/endoscope head, which cap or sleeve is provided or equipped with certain functions/functional elements, as a result of which the endoscope can be used not only for exploration and/or as access for therapeutic applications, but also itself as a minimally invasive instrument for performing a surgical procedure. Alternatively, however, it is also provided to integrally equip special endoscopes for very specific medical applications with such capabilities, such special designs then being suitable only for that particular application.

For example, various diagnostic and/or therapeutic procedures require imaging and/or, if necessary, therapeutic techniques on the biliary and/or pancreatic duct as well as the hepatic ducts of the patient. Since the major duodenal papilla, which forms the common outlet of the biliary and pancreatic duct into the duodenum, protrudes laterally into the duodenum, conventional prograde endoscopes (those with a field of view in the longitudinal direction of the endoscope) are unsuitable for such procedures because there is not enough pivot space in the narrow duodenum (diameter 3 to 4 cm) to align their prograde optics device as well as the working channel in a sideways looking position, since a typical bending diameter of such devices is about 12 cm.

In prior art (e.g. US 2010/228086 A), specially manufactured duodenoscopes are known for this purpose, which have a lateral (sideways looking) and retrograde (backward looking) optics device (also called “lateral optics device”) as well as a possibly laterally directed/opening working channel. At the outlet of a working channel of such duodenoscopes, which opens in the longitudinal direction of the shaft, a so-called Albarran lever is usually provided, which allows a tool guided in the working channel to be specifically guided/diverted to the side (in the radial direction of the endoscope shaft) by pivoting. The laterally oriented arrangement of the functional units on the endoscope head allows imaging and treatment in the region of the duodenum with optimum use of the available space in the patient's intestine.

However, such endoscopes with lateral optics device are very complex and expensive to manufacture and have therefore so far been developed and manufactured as reusable devices. The curved working channel of such endoscopes as well as the design of the Albarran lever which is complex and has many undercuts have proven to be sterilizable in practice, but the sterilization process has proven to pose too much material fatigue on the sensitive devices. Therefore, in practice, the devices are merely disinfected after a procedure. As a result, a bacterial lawn (biofilm) can remain in the working channel and/or the auxiliary channel of the endoscope after a procedure. If this biofilm is then peeled off during a subsequent procedure, for example because an instrument is pushed through the working channel, this film can enter the bile duct and/or pancreatic duct and cause serious inflammation or even sepsis in the patient.

Furthermore, such apparatus have the disadvantage that navigation in the body with sideways looking endoscopes is generally rather difficult, since neither the optics device nor the working channel can be directed in a prograde direction in the narrow duodenum. In order to achieve a forward view with such endoscopes, a bending of the “deflecting” (actively curvable endoscope shaft portion) immediately in front of the endoscope head by approx. 90° is necessary, which in turn requires more space in the transverse direction of the endoscope, which is only available in the stomach. Looking ahead with a 90° bend of the deflecting in the duodenum or other comparatively narrow portions of the gastrointestinal tract would pose the risk of injury to the mucosa of the lumen or even intestinal perforation. Therefore, such side-view endoscopes can only be used for a few, very specific procedures in the region of the duodenum.

The deflecting, arranged in the distal portion of the shaft, of a prograde (forward-looking) flexible endoscope with a tip that can be angled via the deflecting is usually made up of articulated ring elements that form the support structure of the shaft and are operated and tilted relative to one another via Bowden cables, often called bending control cables. To facilitate insertion into the cavity and prevent the permeation of substances, the ring elements are surrounded by a flexible covering made of a plastic material. In particular, light and image transport cables, channels for fluids or endoscopic working instruments run inside the ring elements. The bending control cables are guided along the outer or inner side of the ring elements. Such flexible endoscopes are disclosed, for example, in U.S. Pat. No. 6,270,453 B1, U.S. Pat. No. 6,482,149 B1 or DE 101 43 966 B4.

The smallest radius that the flexible or articulated portion of the shaft can have is determined by the respective design principle. For example, a joint portion formed with successive ring elements which are each hinged to one another permits only a relatively large bending radius, since the individual ring elements each have only a small tilt angle to the next ring element. If the articulated connection of two elements only allows tilting about one axis, it is necessary for a spatial bending option to arrange elements with tilting axes alternately rotated against one another, so that only every second element can be deflected in a respective desired direction; this again increases the possible minimum bending radius. The area next to the shaft end is therefore not visible with a forward-looking optics device arranged in the endoscopic tip, which means for the colon region, where lateral eversions occur physiologically, that polyps or tumors up to a size of about 1 cm can be overlooked because they lie laterally in said eversions.

In this context, it should be noted that a deflecting as described above is provided and designed for the purpose
of facilitating the insertion of an endoscope, for example into a patient's colon, in that the deflecting can be actively adapted to the curvatures of the colon, and
of allowing the endoscope to be brought close to the intestinal wall.

Accordingly, the radius of curvature of such a deflecting is comparatively large.

In prior art, as described for example in DE 10 2013 222 279 A1 or DE 10 2012 220 578 A1, endoscopes are known which have an optics device that can be pivoted separately from the rest of the endoscope head, and which can look in both prograde and lateral directions. However, such endoscopes do not have a working channel (i.e., they are used purely for diagnostic purposes) or have a fixed working channel in prograde direction and are thus not suitable for the typical applications of duodenoscopes, which require a sideward/radially oriented working channel or a deflection device.

In summary, it can be said that with the duodenoscopes known from prior art so far, the user is not able to perform minimally invasive procedures in prograde direction as well as in the lateral/backward-looking vicinity of the endoscope and to detect polyps or tumors (colon examination).

### SUMMARY

In view of the above described disadvantages of the state of the art, it is the object of the present invention to provide a finely adjustable (compared to a known deflecting) pivot mechanism for an endoscope head with a comparatively small pivot radius, which allows the observation and the integrated minimally invasive surgical treatment of the vicinity surrounding the endoscope tip, especially in the context of duodenum endoscopy.

The solution of the above object provides in principle for the provision of a plurality of technical measures which in their combination lead to significantly smaller pivot radii compared to known deflecting structures. Specifically, the minimum possible pivot or curvature radius of the (finely adjustable) pivot mechanism of the invention according to a first aspect of the invention depends essentially on the shape of at least one or more segments (of the endoscope head) of the pivot mechanism which can be pivoted/angled relative to one another, as well as on the positioning of the working channel within the respective segment. The decisive factor is that the segment shape is as flat/thin as possible in a bending direction area and as thick as possible in a diametrically opposite support area, so that two adjacent/adjoining segments can be pivoted/tilted relative to one another in the predetermined bending direction by a comparatively large angle and that this pivoting/tilting is not impaired at all or impaired as little as possible by the working channel of the endoscope. The latter is achieved by placing the working channel off-center in the thick area of the respective segment instead of arranging it centrally.

Stated in other words, according to the first aspect of the present invention, at least one segment of the (finely adjustable) pivot mechanism according to the invention, which segment is directly adjacent to the endoscope head (having an optics device as well as a working channel exit) or to a part of the endoscope head, and thus can be quasi associated with the endoscope head (as a component thereof), is formed as a disk/plate which is substantially round in plan view and substantially wedge-shaped in side view. Accordingly, the/each disk/plate has a flat circumferential portion as seen in side view, which continuously thickens (in a wedge shape) towards the diametrically opposite circumferential portion and reaches its plate thickness maximum at this opposite circumferential portion. This plate thickness maximum is maintained over a (small) partial circumferential portion, resulting in a contact surface (small compared to the wedge-shaped portion) that is oriented essentially perpendicular to the plate center axis. As seen in plan view, the working channel is formed to be decentral at least partially in this partial circumferential section of maximum plate thickness.

In prograde alignment of the endoscope shaft or of the finely adjustable pivot mechanism according to the invention, the endoscope head and the above-described plate segment and/or the above-described plate element and another plate element of the pivot mechanism adjacent thereto with preferably the same structure are supported on one another at the (small) partial circumferential sections, as a result of which this prograde alignment can be kept stable. If an actuating mechanism (e.g. a Bowden cable) is activated, the endoscope head and the one plate segment described and/or the one plate segment described and the described further plate segment adjacent thereto fold down towards one another, as a result of which the flat portions of the segment(s) approach and ultimately touch one another. The maximum folding/pivot angle corresponds to the spanned wedge angle between the respectively opposing segments, which is not or only marginally restricted by the working channel since it is formed at least partially outside the wedge shape area.

Accordingly, in a preferred embodiment, the endoscope according to the invention has an elongated main body and, at its distal end portion (facing away from the user), a distally arranged endoscope head (usually/preferably distal to an actively curvable so-called “deflecting zone”) or a distal end cap provided with at least one optics device for image transmission, a lamp and a working channel for guiding tools and/or for the flow of media. In addition, the endoscope according to the invention has the internal (additional) folding or pivot mechanism which is different from the generally known “deflecting” (and, if present, is spaced in distal direction therefrom) and which is configured to pivot at least a portion of the endoscope head from a prograde alignment into a lateral or sideward or retrograde alignment, i.e., about a pivot axis transverse to the longitudinal direction of the endoscope, in particular continuously variable but possibly also in a stepwise manner. At least the optics device and the working channel are directly or indirectly coupled to the pivot mechanism in such a way that they are pivoted together with the pivot mechanism or with the endoscope head portion which can be pivoted by the former. In other words, the endoscope head according to the invention forms a pivotable optics device and working channel unit, so that an endoscope equipped/formed with it can be used in both prograde and lateral/retrograde alignment. In the context of this application, the word “optics device” stands collectively for all imaging systems known to the state of the art in the field of endoscopy, such as, for example, a module consisting of CMOS or CCD chips comprising a lens and a lamp (LED) in the endoscope head or the use of light guides, etc. It is irrelevant here whether the basic position of the pivot mechanism is prograde or lateral. To implement the idea of the invention, it is only important that the pivot mechanism is able to assume both alignments, so that an endoscope according to the invention combines the functions of a prograde endoscope and an endoscope with lateral optical device.

According to the first aspect of the invention, the pivot mechanism may comprise a number (or a plurality) of axially consecutive segments which are actively adjustable relative to one another in their angle by means of at least one actuating element, which define in the axial direction at least one (working) channel for the passage of minimally invasive surgical instruments, irrigation media, supply lines and the like. In this case, the segments can be designed as wedge-shaped cylindrical sections (plates) with end faces aligned with one another in a wedge shape, as a result of which each cylindrical section has a cylindrical envelope portion of minimum axial length (minimum plate thickness) and an opposite cylindrical envelope portion of maximum axial length (maximum plate thickness), wherein two directly adjoining segments are aligned in each case relative to one another such that they are axially supported or rest upon each other at their respective cylindrical envelope portions of maximum axial length, resulting in a hinge or joint contact on the support or abutment area. Due to the wedge shape of the segments and the axially aligned or adjacent arrangement of the respective adjacent widest portions of the wedge shape, the relative center of rotation of the respective adjacent segments can be moved as far as possible into the peripheral region of maximum plate thickness, as a result of which a comparatively large (wedge-shaped) clearance is created between the tips of the wedge shapes and a relatively large possible tilt angle between the individual segments can be achieved. This results in a comparatively small bending radius of the pivot mechanism, so that, in contrast to prograde endoscopes equipped only with a conventional deflecting, also the lateral vicinity can be treated and observed by an endoscope with such a folding mechanism.

Preferably, the actuating element for the angular adjustment between the segments can be designed in the form of at least one tensile element, which pulls the wedge tips of the segments together by applying a tensile force to the most distally arranged wedge-shaped segment, thus causing the folding/bending of the pivot mechanism. Preferably, at least one channel for the at least one tensile element can be formed axially through the respective segments so that it runs in an inner lumen of the segments. Preferred designs for the tensile element are, for example, cable pulls. It is particularly preferred that the tensile element is designed as a Bowden cable, which is at least indirectly anchored or supported at the most distally and the most proximally arranged segment and braces them against one another for folding/bending the pivot mechanism.

The return movement to the straight position can take place, for example, via shear stiffness (as with a shear-resistant Bowden cable) or via the inherent elasticity of the connection of the wedge segments or also with incorporated spring elements. If necessary, this return movement can also go slightly beyond the straight-ahead position in order to extend the viewing range and to pretension and thus stiffen the endoscope tip in the straight-ahead position.

According to a preferred aspect of the invention, a strap brake may be provided to fix the position of the pivot mechanism, comprising a preferably shear-resistant brake strap fixed to the most distal wedge-shaped segment or to the endoscope head and extending through the regions of minimum axial length (wedge tips) of the segments in the longitudinal direction of the endoscope. The strap brake may preferably have a brake shoe located proximal to the wedge-shaped segments, which is engageable with the brake strap to hold it in place and thus fix the position of the pivot mechanism. Advantageously, a piezoelectric element may be provided for actuating the brake shoe.

According to a second aspect of the invention, which may exist independently or in combination with the first aspect above, the pivot mechanism has at least two, preferably at least three flexible, shear-resistant wires or rods (shafts) which extend within the elongated body (in an peripheral region) of the endoscope along its longitudinal direction and are movable relative to the main body. Here, the wires or rods are articulated on a proximal side of the endoscope head so as to be able to tilt/rotate.

In other words, the endoscope according to the second aspect of the present invention has an endoscope shaft, optionally an actively operable deflecting of known design arranged distally on the endoscope shaft, and an endoscope head at the distal shaft or deflecting end. Furthermore, at least two, preferably three resiliently flexible, shear-resistant wires/rods are mounted in the endoscope shaft and (if present) in the deflecting, which are hinged/fixed distally on the endoscope head or an endoscope head part. The wires/rods are intended and designed to the effect or are mounted and supported in such a way that they can only move the endoscope head or the corresponding (distal) endoscope head part relative to the immediately axially adjacent (proximal) endoscope segment (corresponding to a further proximal endoscope head part, so to say), wherein they are preferably selectively (independently of one another) moved back and forth (and thus are extended or retracted from the proximal endoscope head part), as a result of which only the endoscope head or the distal endoscope head part is moved/swiveled relative to the shaft (which is not actuated in this process).

This means that the wires/rods can be advanced/adjusted starting from the proximal side beyond the endoscope shaft. By advancing the individual wires or rods axially beyond the shaft by different distances, it is possible to achieve different swivel positions only of the endoscope head or distal head part with respect to the shaft or proximal head part, similar to the principle of a tripod with three (or more) legs which are adjustable in their length. For the purpose of achieving a sealing between the (tubular) end portion of the shaft or proximal head part and the endoscope head or distal head part in the advanced/folded position, an expandable covering, preferably made of polyurethane or silicone, a bellows or the like can be used, for instance.

Preferably, such an embodiment can be designed with at least three flexible wires with a robotic control, i.e. the wires can be operated e.g. with linear motors, hydraulic pistons or the like and by means of a corresponding control software. It is also possible to implement the operation of the pivot mechanism or the feed control of the individual wires by means of manual control elements such as rotary knobs or the like.

According to a third aspect of the invention, which may exist independently of or in combination with the first and/or second aspect, an additional fine adjustment device or fine angular adjustment may be provided between the pivot mechanism or the endoscope shaft (or deflecting) and the endoscope head/endoscope head part arranged distally to it, for allowing precise alignment of the working channel exit or a minimally invasive instrument guided therethrough.

According to a preferred exemplary embodiment of the invention, a hydraulic actuator may be provided for fine angular adjustment, which is arranged between the pivot mechanism and the endoscope head and which may preferably be arranged in a peripheral region of the endoscope head near the outer radius of the pivot movement performed by the pivot mechanism.

According to a further preferred exemplary embodiment of the invention, the fine adjustment may comprise a magnetizable flexion spring biased into a maximum tilted position (about 5°-10° relative to the prograde alignment). Further, the fine adjustment may include a solenoid which is disposed proximal to the flexion spring and can be powered with electric current to pull the flexion spring into the prograde alignment against its biasing direction.

According to a further preferred exemplary embodiment of the invention, the fine adjustment may have at least two wedge rings (wedge-shaped ring segments) that can be rotated relative to each other about the central axis of the endoscope, wherein the distal end face of the distal wedge ring can be adjusted with respect to the proximal side face of the proximal wedge ring by relative rotation of the two wedge rings. This can be achieved, for example, by means of a resiliently flexible, torsionally stiff drive shaft. The axial cohesion of the wedge rings can be achieved, for example, by means of a tension spring or with a form fit by means of a ball-and-socket arrangement.

According to a further preferred embodiment of the invention, the endoscope head can be spring-loaded in a position inclined by 5° to 10° to the longitudinal axis of the endoscope for fine adjustment, and a Bowden cable can be provided in the peripheral region of the outer bending radius of the fine adjustment, by means of which the head can be pulled into a prograde alignment in order to accomplish a fine adjustment in said angular range of 5° to 10°.

According to a further preferred exemplary embodiment of the invention, a piezo-based flexural transducer can be provided for fine adjustment and as part of a joint connection between the pivot mechanism and the endoscope head. By varying the voltage (which is provided via a corresponding line from a base station), the bending of the piezo-based flexural transducer and thus the tilt angle of the head support can be finely adjusted. Here, it is advantageous that the piezo-based flexural transducer does not require any power consumption when holding its position and therefore does not heat up.

### BRIEF DESCRIPTION OF THE DRAWING FIGURES

FIG. 1A is an illustration of a general endoscope design as is common in prior art;

FIG. 1B is an illustration for illustrating a field of application of an endoscope having a pivot mechanism of the invention according to a first aspect of the invention;

FIG. 2A is an illustration of a first embodiment of the pivot mechanism of the invention according to FIG. 1B in a front view;

FIG. 2B is an illustration of the first embodiment of the pivot mechanism of the invention according to FIG. 1B in a side view;

FIG. 3A is a perspective illustration of a pivot mechanism of the invention in a prograde alignment according to a second aspect of the invention;

FIG. 3B is a perspective illustration of the pivot mechanism of the invention according to FIG. 3A in an alignment pivoted by about 45°;

FIG. 4 is a perspective illustration of a fine adjustment or fine angular adjustment according to a third aspect of the invention;

FIG. 5A is a perspective illustration of a fine adjustment or fine angular adjustment according to a preferred modification;

FIG. 5B is a side view of the fine adjustment or fine angular adjustment according to FIG. 5A;

FIG. 6A is a side view illustration of a fine adjustment or fine angular adjustment according to a further modification in a prograde alignment;

FIG. 6B is a side view of the fine adjustment or fine angular adjustment according to FIG. 6A in an angled alignment;

FIG. 7A is a side view illustration of a fine adjustment or fine angular adjustment according to a further modification in a prograde alignment;

FIG. 7B is a side view of the fine adjustment or fine angular adjustment according to FIG. 7A in an angled alignment;

FIG. 8A is a side view illustration of a fine adjustment or fine angular adjustment according to a further modification in a prograde alignment; and

FIG. 8B is a side view illustration of the fine adjustment or fine angular adjustment according to FIG. 8A in an angled alignment.

### DETAILED DESCRIPTION

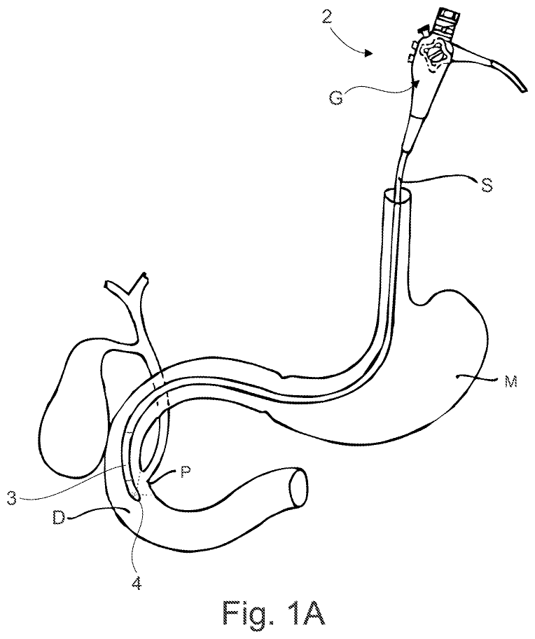

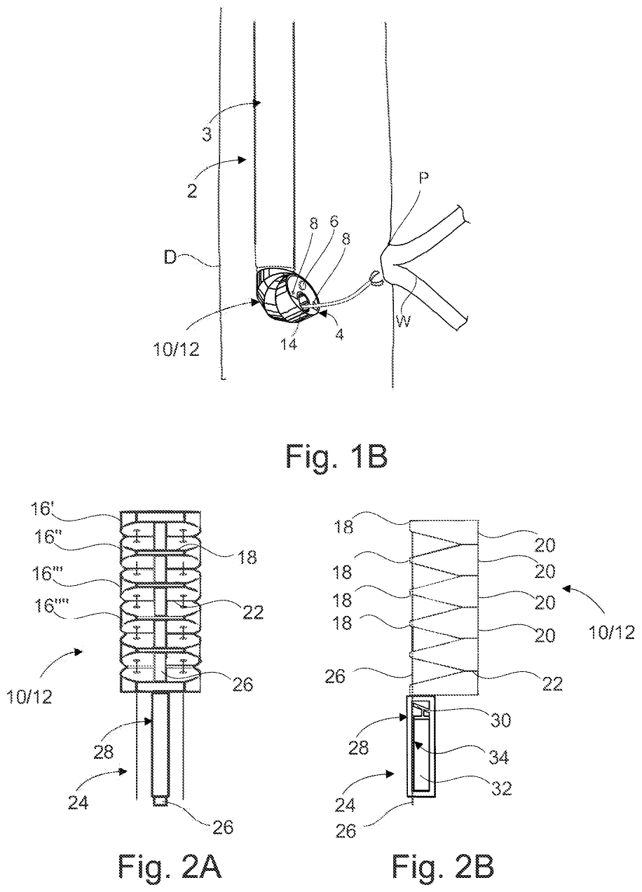

FIG. 1A shows a conventional prior art endoscope design and is used to explain some of the terms employed below. The endoscope 2 has a handle part or operating part G at its proximal end facing the user, by means of which the handling by the user takes place. Distally adjacent to the handle part G is an elongated main body of the endoscope 2, which can be divided in the direction from proximal to distal into a series of consecutive portions, each with a different functionality: a shear-resistant and passively resilient flexible shaft S, an actively curvable distal shaft portion 3 (also called deflecting portion) and a distal, usually rigid end cap also called endoscope head 4. The endoscope 2 shown in FIG. 1A is inserted into the duodenum D via the esophagus and the stomach M, so that the endoscope head 4 is located in the area in front of the major duodenal papilla.

FIG. 1B is used to explain a preferred field of application of an endoscope according to the invention comprising a pivot mechanism. In addition, FIG. 1B shows a first preferred embodiment of such a pivot mechanism in an angled state.

FIG. 1B schematically shows the major duodenal papilla (P), which is located in the posterior (dorsal) descending part (Pars descendens) of the duodenum (D) and is relatively difficult to access due to the tortuous geometry of this system. The available space in the area of the duodenum (D) is very limited, which makes interventions at the major duodenal papilla (P) impossible with conventional, prograde endoscopes, since an appropriate angling of the endoscope tip toward the interventional section would not leave enough distance to the lumen of the duodenum (D) for proper imaging. In addition, such angling would be accompanied by the risk of perforation in the region of the duodenum due to the high bending radius of conventional devices.

For this reason, the duodenoscopes initially mentioned are known to prior art, which have a fixed optics device looking sideways or backward as well as a correspondingly aligned working channel in order to make optimum use of the available space. However, such duodenoscopes have the disadvantage that they are fixed in their lateral/retrograde alignment of the optics device and of the working channel. This complicates the general navigation within the patient, on the one hand, and makes such endoscopes inflexible in their application possibilities, on the other hand. In other words, they are expensive specialized devices for a narrowly limited field of application.

A basic idea of the present invention is therefore to provide, in addition or as an alternative to a known deflecting, a pivot mechanism associated with the endoscope head for the endoscope head or an endoscope head part, by means of which the endoscope head (head part) together with the optics device arranged thereon and the working channel exit of the endoscope can be pivoted by at least 90° without requiring a large bending radius for this purpose. In particular, a finely adjustable pivoting movement should be made possible, for example, to facilitate intubation of the major duodenal papilla. At the same time, a multifunctional device is to be provided, with the aid of which the majority of common applications in the area of the gastrointestinal tract can be performed.

For this purpose, the endoscope 2 shown in FIG. 1B has a first exemplary embodiment of a pivot mechanism 10 according to the invention, which in the perspective illustration is tilted by 90° with respect to a prograde/straight looking orientation in order to perform an intervention on the major duodenal papilla P. At the distal end of the pivot mechanism 10, an endoscope head or at least a part 4 of the endoscope head is arranged, which comprises or forms various functional units such as an optics device 6; a lamp 8 and a working channel 14. For the sake of clarity, only the above-mentioned most necessary functional units are shown in FIG. 1B, but it should be understood that an endoscope head according to the invention may additionally have various other functional units known from prior art, such as cleaning nozzles for an objective of the optics device 6, suction channels, etc., in the area of the depicted part 4.

The pivot mechanism 10 shown in FIGS. 2A and 2B in a front and a side view, respectively, has a main body 12 with a number of segments 16 (16′, 16″, 16′″ and 16″″) arranged sequentially in the axial direction of the endoscope 2. It should be noted at this point that at least the distally last segment 16′ (or all further segments) can also be regarded as a proximal part of the endoscope head 4, since they differ in their design and function from the rest of the shaft and from the optional, proximally arranged deflecting (if present).

The individual segments 16 basically have a wedge-like shape, i.e. their distal and/or proximal end faces converge at an acute angle and meet (as seen in side view) in a wedge tip 18. Accordingly, the axial extension of the individual segments 16 is greatest in a (diametrically opposite) portion 20 facing away from the wedge tip 18.

As can be taken in particular from FIG. 2B, the two end faces in the region of maximum segment thickness are flattened (aligned substantially parallel to each other) over a pitch circle section which is small compared to the wedge section (between the tip and the maximum segment thickness), thus forming a support/abutment surface for supporting two adjoining segments in prograde shaft alignment.

Preferably, in the illustrated embodiment, the individual segments 16 are further circular cylindrical in shape to provide a circular cross-section along the entire endoscope 2. As can be clearly seen in FIG. 2B, the wedge tips 18 and the portions 20 facing away from them are in alignment in the upright/straight position of the pivot mechanism 10. As a result, the pivot mechanism 10 can be angled unidirectionally.

Furthermore, it can be clearly seen in FIG. 1B that the working channel formed in the segments 16 is designed to be decentral at least in part in the area of the segments of maximum segment thickness, i.e. as close as possible to that circumferential portion of the respective segment which is diametrically opposite the segment tip.

In the upright/prograde configuration of the pivot mechanism 10 shown in FIG. 2A, the portions 20 of maximum segment thickness facing away from the wedge tips 18 respectively adjoin one another and form support or abutment areas/surfaces 22 around which or at which the individual segments 16 can be tilted relative to one another. Due to the wedge shape of the segments 16, sawtooth-like open spaces are formed towards the wedge tips 18 in the main body 12 of the pivot mechanism 10 in the upright position as shown in FIG. 2. The segments 16, which can be tilted about the support or abutment areas 22, can thus be folded towards one another, reducing these open spaces, until the respective proximal and distal end faces of adjacent segments 16 abut one another in a maximally curved position, as shown in FIG. 1B. Due to the fact that the wedge tips 18 have a relatively small axial extension of less than 1 mm in this case, a position folded by 90° can be achieved with a comparatively small inner bending radius. The small inner bending radius has the advantage that the optics device 6 and the lamp 8 do not protrude far beyond the outer circumference of the endoscope in said folded position (see FIG. 1B).

In order to effect the bending or folding described above, Bowden cable channels (not shown in detail here) are provided in the segments 16, in which Bowden cables (also not shown here) are guided. These are configured to apply a tensile force to the most distally arranged segment 16′, thereby generating a torque in the support or abutment areas 22, which causes the pivot mechanism 10 to fold/get curved. Corresponding Bowden cable mechanisms and associated operating elements are sufficiently known in prior art and are not explained in more detail here.

Preferably, a flexible or elastic hose inserted into the working channel can be used as the pivot hinge, which connects the loosely disposed segments to one another in a pivotable manner. Alternatively, however, it is also possible to dispense with such a hose and instead put in wires in the hinge area of the segments which hold the hinges together axially, as indicated in FIG. 2A.

In the preferred embodiment shown, the pivot mechanism 10 also has a strap brake 24 that can be used to fix the position thereof. The strap brake 24 according to the embodiment shown in FIGS. 2A and 2B has a shear-resistant, resiliently flexible brake strap 26 that runs along the inner bending radius of the pivot mechanism 10 through the segments 16 in the area of their wedge tips 18. The brake strap 26 is fixed to the segment 16′ supporting the endoscope head 4 and arranged furthest distally. If the pivot mechanism 10 is angled/tilted by means of the Bowden cables, the brake strap 26 moves axially in the proximal direction and passes through a braking member 28 proximally adjacent to the pivot mechanism. The braking member 28 has a brake shoe 30, which is designed as a solid-state joint. In the illustrated example, the brake shoe 30 is realized in the form of an elastic protrusion integrally formed in the region of the braking member 28 and extending obliquely between the radial direction and the distal axial direction of the endoscope 2. The brake strap 26 extends between a free end portion of the brake shoe 30 and a wall portion 34 of the braking member 28. The brake shoe 30 is connected to an axially acting piezoelectric element 32 which, when a voltage is applied thereto, pulls the brake shoe 30 toward proximal into a substantially radial alignment and in this position clamps the brake strap 28 against the wall portion 34, thereby locking the pivot mechanism 10. When the piezoelectric element 32 is extended, the brake strap 28 is released and the pivot mechanism 10 can be moved.

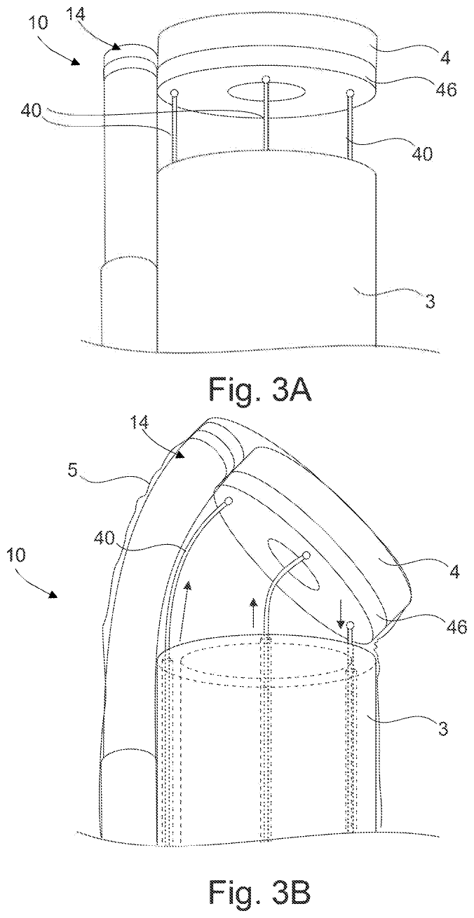

FIGS. 3A and 3B illustrate a second preferred embodiment of a pivot mechanism 10. In this embodiment, a support disk 46 is preferably provided as a distal part of the endoscope head, having its distally facing surface provided with a functional support 4 (for receiving, for example, an optics device and an illumination unit as well as a working channel exit) of the endoscope head. The support disk 46 is connected via a number (here three) of push-resistant, resiliently flexible drive shafts 40 (wires/rods, which can be considered as the proximal part of the endoscope head) to an actively curvable portion 3 (deflecting) of the endoscope shaft or, if this is not present, to the endoscope shaft itself. The drive shafts are each articulated/fixed in a peripheral region of the support disk 46, on its proximally facing side. By controlling the feed of the individual drive shafts 40 or by adjusting their respective protrusion beyond the distal edge (distal end face) of the actively curvable portion 3 or of the endoscope shaft, different tilt positions of the support disk 46 with respect to the distal end face of the deflecting/endoscope shaft can be set, as can be seen in FIG. 3B. The use of three drive shafts 40 according to the illustrated preferred embodiment has the advantage of avoiding static overdeterminations. In the illustrated embodiment, the drive shafts 40 are designed with robotic control, i.e. they are operated by means of small motors (not shown) and by software control. An operation of the pivot mechanism or a control of the feed of the individual wires by means of manual control elements such as rotary knobs or similar handling elements can be implemented just as well. For the purpose of achieving a sealing between the actively curvable portion 3 or the endoscope shaft and the endoscope head part 4 or the support disk 46 in the maximum advanced position shown in FIG. 3B, an expandable covering 5, preferably made of polyurethane or silicone, a bellows or the like is provided.

In the example shown in FIGS. 3A and 3B, the working channel 14 is arranged radially outside the main body of the endoscope (outside the shaft S, the deflecting 3 and the endoscope head 4), i.e. as far out as possible with respect to the intended direction of pivoting. This offers the advantage that when the pivot mechanism 10 is angled, as shown in FIG. 3B, smaller folding or pivoting radii can be achieved with sufficiently large radii of curvature of the working channel (just like in the exemplary embodiment according to the first aspect of the invention described above), which in turn simplifies the process of pushing a minimally invasive tool through it.

Conventional endoscopes for interventions on the major duodenal papilla (so-called duodenoscopes) have an actively operable deflection lever (also called Albarran lever) arranged in the distal area of a working channel exit aligned in prograde manner, by means of which minimally invasive instruments, guide wires and the like advanced through the working channel can be selectively deflected, whereas the alignment of the optics device is fixed and usually cannot be changed.

The pivoting mechanisms 10 of the invention according to the exemplary embodiments described above make the aforementioned Albarran lever obsolete, since minimally invasive instruments, guide wires and the like together with the optics device can be deflected per se in a very small space by the alignment capability of the working channel exit. As explained above, it is advantageous if the working channel 14 is arranged on the outer side or radially on the outside between the shaft S and the covering 5 in the area of maximum segment thickness, so that it can be located in the outer radius of the bend when the pivot mechanism 10 is bent and thus has as little influence as possible on the maximum possible folding path. Depending on the selected embodiment of the pivot mechanism 10, however, an additional fine adjustment of the pivoting/bending can be advantageous, e.g. to intubate the major duodenal papilla in a targeted manner.

According to a further aspect of the invention, which may exist independently, a fine adjustment device (fine adjustment or fine angle adjustment) 52 may therefore be provided in addition to the aforementioned pivoting member 10, which is provided according to the preferred embodiment between the pivoting member 10 and the endoscope head 4. The following fine adjustments are each shown by way of example in the arrangement on a segment 16′ according to the first embodiment of the pivoting member shown in FIGS. 1, 2A and 2B. Of course, a combination with the other embodiments for a pivoting member is equally possible and envisaged.

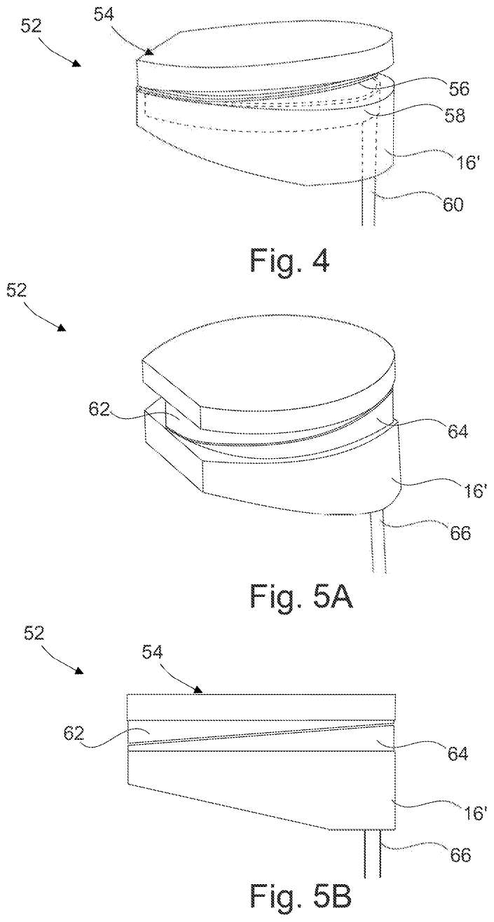

FIG. 4 shows an embodiment of the invention comprising a fine adjustment 52. The fine adjustment shown has a disk-shaped or plate-shaped head support 54, which is biased by means of a magnetizable flexion spring 56 into a maximum tilted position (approx. 5°-10° relative to the prograde alignment). A solenoid 58 disposed proximal to the flexion spring 56 in a segment 16′ of the pivoting member 10 is connected to a base station (not shown) via a power supply 60. By varying the current flowing through the solenoid 58, the head support 54 can be pulled toward the solenoid 58 in opposition to the bias. Preferably, a separate fixation element (not shown) can be used to hold the finely adjusted alignment in order to avoid excessive coil heating.

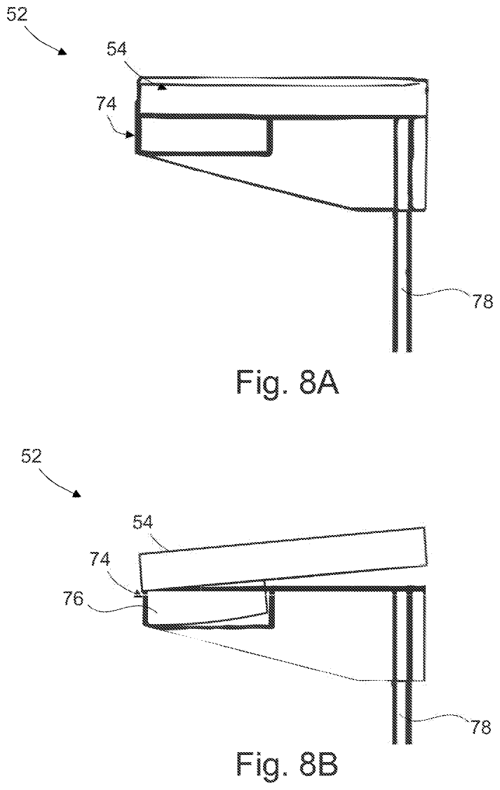

FIGS. 5A and 5B show a further embodiment of the invention comprising a fine adjustment 52. In the further embodiment of the fine adjustment 52 shown, the head support 54 is connected to a segment 16′ of the pivoting member 10 via two wedge rings 62, 64 which can be rotated relative to each other about the central axis of the endoscope. In FIGS. 8A and 8B, the wedge rings 62, 64 are shown in an orientation symmetrical in cross-section, which causes the head support 4 to be positioned straight. If, for example, the distal wedge ring 62 is rotated relative to the proximal wedge ring 64, the distal end face of the distal wedge ring (and thus the head support 54) tilts relative to the longitudinal axis of the endoscope. In the example shown, the relative rotation is effected by means of a resiliently flexible, torsionally stiff drive shaft 66, the distal end region of which is provided with a pinion, not shown, which interacts with a ring gear on the distal wedge ring 62. The axial cohesion of the wedge rings 62, 64 can be achieved, for example, by means of a tension spring, spring U-bolt, ball-and-socket arrangement or comparable form-fit arrangements. Alternatively, the drive shaft 66 can be fixed to the distal wedge ring 62 in tensile-proof manner in order to pull the latter toward the proximal wedge ring 64. This embodiment has the advantage that a very good rigidity of the finely adjusted positioning can be achieved. The arrangement described above can also be doubled, i.e. at least four wedge rings can be provided which are alternately fixed or rotatable relative to one another and which can be rotated relative to one another by means of two drive shafts 66. Such an arrangement allows pivoting in all directions. The free interior space of the wedge rings 62, 64, which is freely visible in FIG. 8B, provides sufficient space for channels and lines.

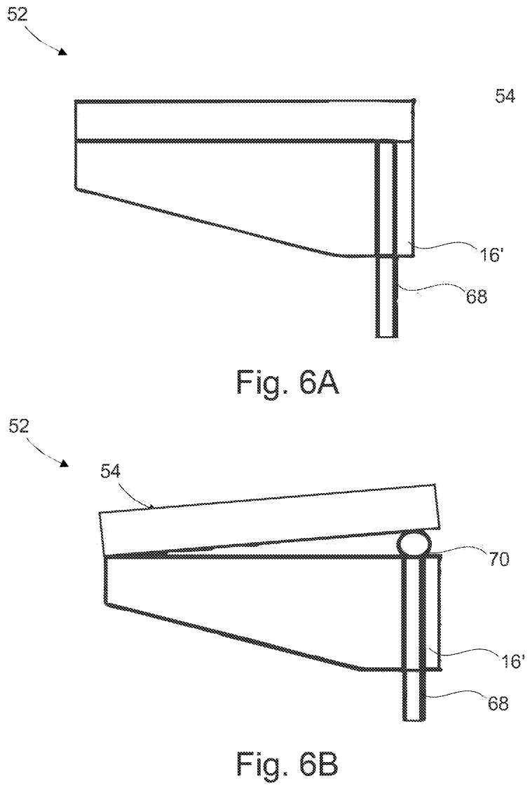

FIGS. 6A and 6B show a further embodiment of the invention comprising a fine adjustment 52, in which the head support 54 is held in the prograde position by spring pretension. On the circumferential side in the region of the inner bending radius of the pivot mechanism 10, the head support 54 is pivotably arranged on the segment 16′. On an opposite peripheral portion, a hydraulic hose 68 extending in the axial direction of the endoscope is provided, which ends with its distal end below the head support 54. At said distal end of the hydraulic hose 68, a balloon or piston element 70 is arranged which, when pressurized, accomplishes a variable tilting of the head support 54. Such a system offers the advantage of good stiffness of the finely adjusted position.

In addition, a water-filled hydraulic hose 68 has the advantage of good biocompatibility, which minimizes risks to the patient in the event of a malfunction. In combination with the first embodiment of the pivot mechanism 10 (FIGS. 1, 2A and 2B) comprising wedge-shaped segments 16, the hydraulic hose 68 can be used as a tilt joint for the segments 16 at the same time and thus does not require additional space.

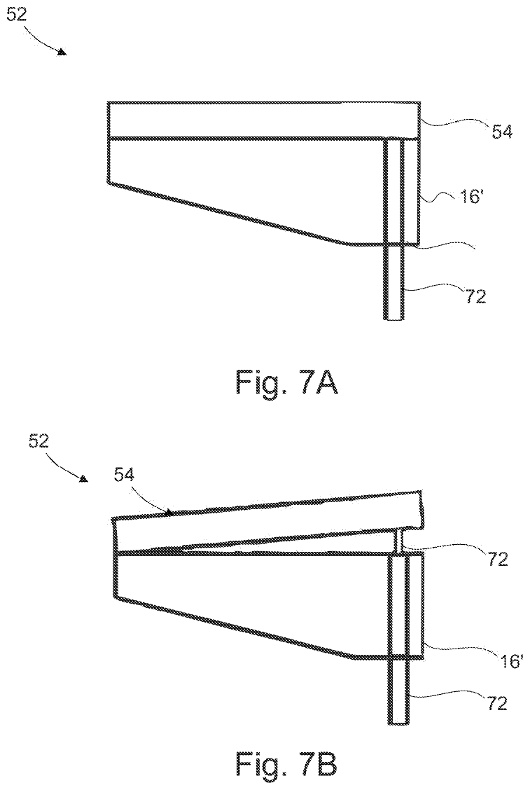

FIGS. 7A and 7B show a further embodiment of the invention comprising a fine adjustment 52, which functions similarly to the immediately aforementioned embodiment of the fine adjustment; here, however, the head support 54 is preloaded in its end position (e.g. inclined 5° to 10° relative to the longitudinal axis of the endoscope). A Bowden cable 72 arranged in the peripheral region of the outer bending radius of the pivot mechanism 10 is used to pull the head support 54 against the pretension in the direction toward the wedge-shaped segment 16′. Due to the Bowden cable 72, such a fine adjustment 52 also has good stiffness and no approval problems for the medical field, as the use of Bowden cables is proven in endoscopy. In combination with the first embodiment of the pivot mechanism 10 (FIGS. 1, 2A and 2B) comprising wedge-shaped segments 16, the Bowden cable 72 can also be used as a tilt joint for the segments 16 and thus does not require any additional space.

FIGS. 8A and 8B show a further embodiment of the invention with a fine adjustment 52. In this embodiment, a piezo-based flexural transducer 74 arranged in the area of the inner bending radius of the pivot mechanism is used as a joint connection between the pivot mechanism 10 (the segment 16′) and the head support 54. In the prograde normal position, the head support 54 is held in position by the piezoelectric element 76 of the piezo-based flexural transducer 74. By varying the voltage (transmitted via line 78), the bending of the piezo-based flexural transducer 74 and thus the tilt angle of the head supports 54 can be finely adjusted. This has the advantage that the piezo-based flexural transducer 74 does not require any power consumption when holding its position and consequently does not heat up. This design variant also advantageously manages without any spring pretension.

1. An endoscope having comprising:
an endoscope shaft comprising a distal end portion;
an endoscope head arranged on the distal end portion of the endoscope shaft, the endoscope head or a distal part of the endoscope head having or supporting at least one optics device for imaging; and
a working channel that extends integrally within the endoscope in a longitudinal direction, the working channel having a working channel exit arranged or formed in the endoscope head or in the distal part of the endoscope head,
wherein
a pivot mechanism arranged between the distal shaft end portion of the endoscope shaft and the endoscope head or the distal part of the endoscope head and configured to hold the endoscope head or the distal part of the endoscope head in a foldable or bendable manner, for which the pivot mechanism has at least one segment which defines a disk or plate which is substantially round in plan view and substantially wedge-shaped in side view, said disk or plate having a flat circumferential portion when seen in side view, and which thickens towards a diametrically opposite circumferential portion to obtain a maximum plate thickness maximum at said opposite circumferential portion, which extends over a partial circumferential portion of the plate segment, resulting in a segment support or abutment surface which is oriented substantially perpendicular to the plate central axis and wherein, as seen in plan view, the working channel is formed to be decentral at least partially in said partial circumferential section.

2. The endoscope according to claim 1, wherein the pivot mechanism has a plurality of axially successive segments which are actively adjustable relative to one another in their angle by at least one actuating element, the plurality of axially successive segments being formed as the wedge-shaped plates having end faces oriented in a wedge-shaped manner relative to one another, as a result of which each plate is given a cylindrical envelope portion of minimum axial length and an opposite cylindrical envelope portion of maximum axial length, wherein two directly adjoining segments of the plurality of axially successive segments are aligned in each case relative to one another such that they are axially supported or rest upon each other at their respective cylindrical envelope portions of maximum axial length, resulting in a hinge or joint contact on the segment support or abutment surface.

3. The endoscope according to claim 2, wherein the cylindrical envelope portion of minimum axial length of each plate has an axial length of less than 2 mm.

4. The endoscope according to claim 1, wherein the pivot mechanism has at least two wires, Bowden cables or rods that are resiliently flexible, shear resistant, supported in the endoscope shaft, and distally articulated or fixed on the endoscope head or a distal part of the endoscope head, which is retained on the endoscope shaft only via the wires, Bowden cables or rods, the wires, Bowden cables or rods being provided and configured to be able to move only the endoscope head or the distal endoscope head part relative to the directly and axially proximally adjoining endoscope segment or endoscope head part, wherein the wires, Bowden cables or rods are pushed back and forth such that only the endoscope head or the distal part of the endoscope head is moved relative to the shaft.

5. The endoscope according to claim 1, wherein the endoscope further comprises a fine adjustment device separate from the pivot mechanism, the fine adjustment device allowing a more precise alignment of the working channel exit.

6. The endoscope according to claim 5, wherein the fine adjustment device is a hydraulically actuated actuator to which a pressure can be applied via a hose to bring about an inclination of the endoscope head.

7. The endoscope according to claim 6, wherein the fine adjustment device is biased by a spring element into a maximum tilted position of less than 15° with respect to a longitudinal axis of the endoscope and is transferrable against its biasing direction into a prograde alignment by: a solenoid supplied with electrical current, or (b) a pull wire fixed on the endoscope head and to which a tensile force is applied.

8. The endoscope according to claim 5, wherein the fine adjustment device has at least two wedge rings that rest against and slide on each other and are concentrically rotatable relative to one another.

9. The endoscope according to claim 5, wherein the fine adjustment has a piezo-based flexural transducer to which a voltage can be applied via an electrical line.

10. The endoscope according to claim 1, wherein the endoscope is a single-use combined gastroscope-duodenoscope.

11. The endoscope according to claim 1, wherein the distal end portion of the endoscope shaft is actively curvable.

12. The endoscope according to claim 4, wherein the wires, Bowden cables or rods are pushed back and forth selectively as well as independently of one another.

13. The endoscope according to claim 4, wherein the wires, Bowden cables or rods are pushed back and forth such that only the endoscope head or the distal part of the endoscope head is pivoted relative to the shaft.
